(19) United States
(12) Patent Application Publication
(10) Pub. No.: US 2011/0183432 A1
(43) Pub. Date: Jul. 28, 2011

(54) TEST ELEMENT FOR ANALYZING AN ANALYTE PRESENT IN A SAMPLE OF A BODY FLUID, ANALYSIS SYSTEM AND METHOD FOR CONTROLLING THE MOVEMENT OF A FLUID CONTAINED IN A CHANNEL OF A TEST ELEMENT

(75) Inventors: Manfred Augstein, Mannheim (DE); Susanne Wuerl, Mannheim (DE)

(73) Assignee: ROCHE DIAGNOSTICS OPERATIONS, INC., Indianapolis, IN (US)

(21) Appl. No.: 13/008,104

(22) Filed: Jan. 18, 2011

Related U.S. Application Data
(63) Continuation of application No. PCT/EP2009/003425, filed on May 14, 2009.

(30) Foreign Application Priority Data
Jul. 18, 2008 (EP) 08012980.2

(51) Int. Cl. G01N 1/28; G01N 33/48

(52) U.S. Cl. 436/174; 422/68.1; 422/81

(57) ABSTRACT

Embodiments disclosed herein include an analysis system for the analysis of a body fluid sample for an analyte contained therein, comprising a test element and an evaluation device having a measuring station for measuring a measuring variable on the measuring zone of the test element. Also included is a mounting to hold a test element, the test element having an airflow channel with two expansion sections and a narrow section located between these expansion sections. The expansion sections may have a cross-sectional area that increases in comparison to the narrow section in the direction away therefrom. A connection channel is located between the narrow section and the analysis function channel such that an air exchange connection is formed. The airflow channel is located in such a manner that a partial vacuum, which is generated using an airflow flowing through the airflow channel, acts on the analysis function channel.

### CROSS REFERENCE

This application is a continuation of PCT Application Number PCT/EP/2009/003425, filed May 14, 2009, entitled “Test Element for Analyzing an Analyte Present in a Sample of a Body Fluid, Analysis System and Method for Controlling the Movement of a Fluid Contained in a Channel of a Test Element,” which claims priority to EP Application Number 08012980.2, filed Jul. 18, 2008.

### TECHNICAL FIELD

Disclosed herein are embodiments of a test element for the analysis of a body fluid sample for an analyte contained therein and analysis system and method for controlling the movement of a liquid contained in a channel of a test element.

### BACKGROUND

In the field of medical analytics, a fundamental differentiation is made between analysis systems using “wet reagents” and analysis systems using “dry reagents”. Analysis systems which operate with wet reagents employ technically complex, large, line-operated analysis devices for performing an analysis, wherein the analysis devices allow the required manifold movements of the participating elements.

In the analysis systems operating with dry reagents, these reagents are typically integrated in a test element, which can be implemented as a test strip, for example. During the use of these systems, the liquid sample dissolves the reagents in the test element, the reaction of the sample and reagents resulting in a change of a measuring variable. The measuring variable can be measured on the test element itself using optical or electrochemical measuring methods.

Wet analysis systems with high-performance devices allow the performance of multistep reaction sequences (test protocols) which are utilized, for example, by immunochemical analyses, during whose multistep reaction sequence a “bound/free separation” (“b/f separation” hereafter) is utilized. A b/f separation may include a separation of a bound phase and a free phase. Any of numerous test protocols may be utilized for determining manifold analytes, which differ in manifold ways. They share the feature that they utilize complex handling having a plurality of reaction procedures.

A controlled and multistep reaction sequence is normally not utilized with test strips and similar analysis elements. Test elements similar to test strips may not allow precise control of individual reaction procedures. In particular, these test elements may not allow control of the time sequence of the individual reaction procedures. Wet-chemical laboratory systems offer this possibility. However, they may be too large, too costly, and too complex to handle for many applications.

The gaps are to be closed by analysis systems having controllable test elements. These test elements are implemented so that at least one externally controlled liquid transport occurs therein. Utilizing an externally controlled liquid transport includes using an element outside the test element itself. The external control can be based on the application of pressure differences (overpressure or partial vacuum/reduced pressure) or the change of force actions (such as location change and/or acceleration forces). The external control is frequently performed by centrifugal forces, which act on a rotating test element as the function of the velocity of the rotation.

Controllable test elements typically have a housing, which comprises a dimensionally-stable plastic material and at least one analysis function channel, which is enclosed by the housing. The analysis function channel often comprises a sequence of a plurality of channel sections and chambers lying between them, which are widened in comparison to the channel sections. The structure of the channel including the sections and chambers is defined by a profiling of the plastic parts. This profiling is created in particular by injection-molding techniques or hot stamping, under certain circumstances also by lithography methods.

An analysis function channel in this meaning may include any channel which fulfills a liquid transport function in an analysis method performed by utilizing the test element wherein the liquid transport function contributes to the analysis. This function can comprise, for example, the specific (time controlled) transportation of a liquid, in particular a sample fluid, a reagent liquid (containing at least one reagent), a reaction mixture produced by mixing sample and reagent, or a flushing or washing liquid (for example, for washing away excess reaction participants). The specific transportation comprises the functions “stop”, “transport further in a defined direction (“forward direction”)”, “transport in the opposite direction”. Various forces acting in the analysis function channel, in particular the capillary force, the centrifugal force, and gravity, can be used to control a movement of the liquid in the analysis function channel.

Analysis systems having controllable test elements allow a miniaturization of tests, which could only be performed using large laboratory systems. Parallelization of procedures is possible by application of a plurality of, structures (in some cases, substantially identical structures) for parallel processing of identical or similar analyses from one sample or identical analyses from various samples. Additionally, in many current solutions, the test elements can be produced using established production methods and in that they can be measured using known analysis methods. Known methods and products can also be employed in many current solutions with chemical or biochemical components of such test elements. However, many current solutions fail to adequately address issues related to the control of the movement of a liquid inside the test elements.

### SUMMARY

Embodiments disclosed herein include an analysis system for the analysis of a body fluid sample for an analyte contained therein, comprising a test element and an evaluation device having a measuring station for measuring a measuring variable on the measuring zone of the test element. Also included is a mounting to hold a test element, the test element having an airflow channel with two expansion sections and a narrow section located between these expansion sections. The expansion sections may have a cross-sectional area that increases in comparison to the narrow section in the direction away therefrom. A connection channel is located between the narrow section and the analysis function channel such that an air exchange connection is formed. The airflow channel is located in such a manner that a partial vacuum, which is generated using an airflow flowing through the airflow channel, acts on the analysis function channel.

### BRIEF DESCRIPTION

Embodiments of the present disclosure are explained in greater detail hereafter on the basis of embodiments shown in the figures. The special features shown therein can be used individually or in combination to provide embodiments of the present disclosure. The described embodiments are no restriction of the generality of the subject matter defined in the claims. In the figures:

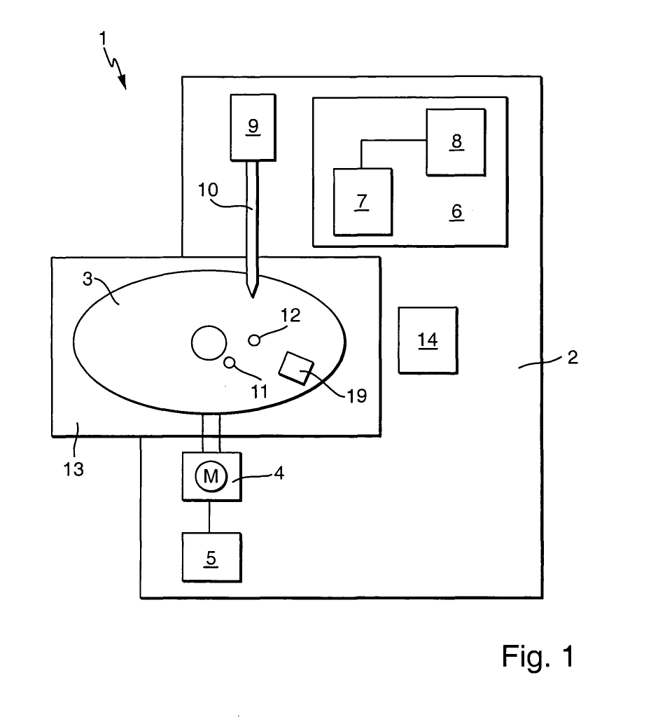

FIG. 1 shows a schematic diagram of the analysis system, according to embodiments of the present disclosure;

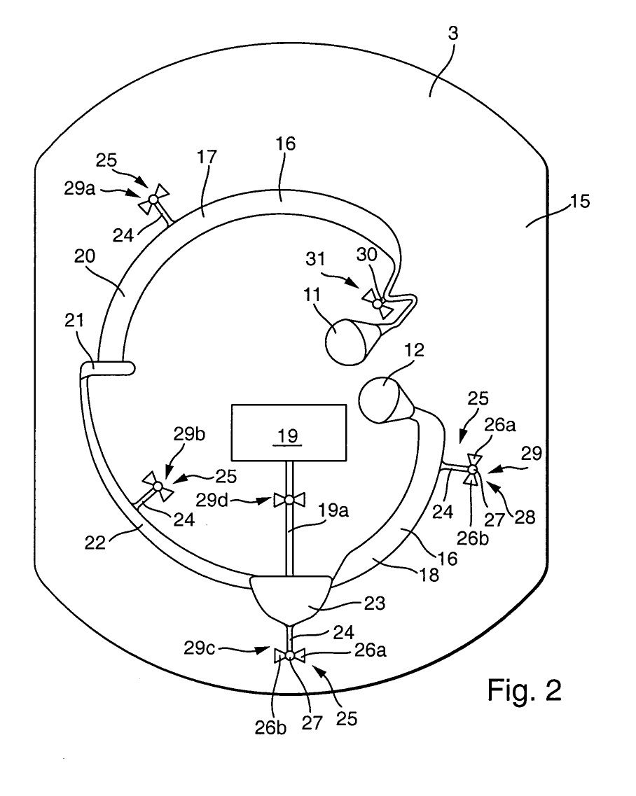

FIG. 2 shows a schematic diagram of a test element, according to embodiments of the present disclosure;

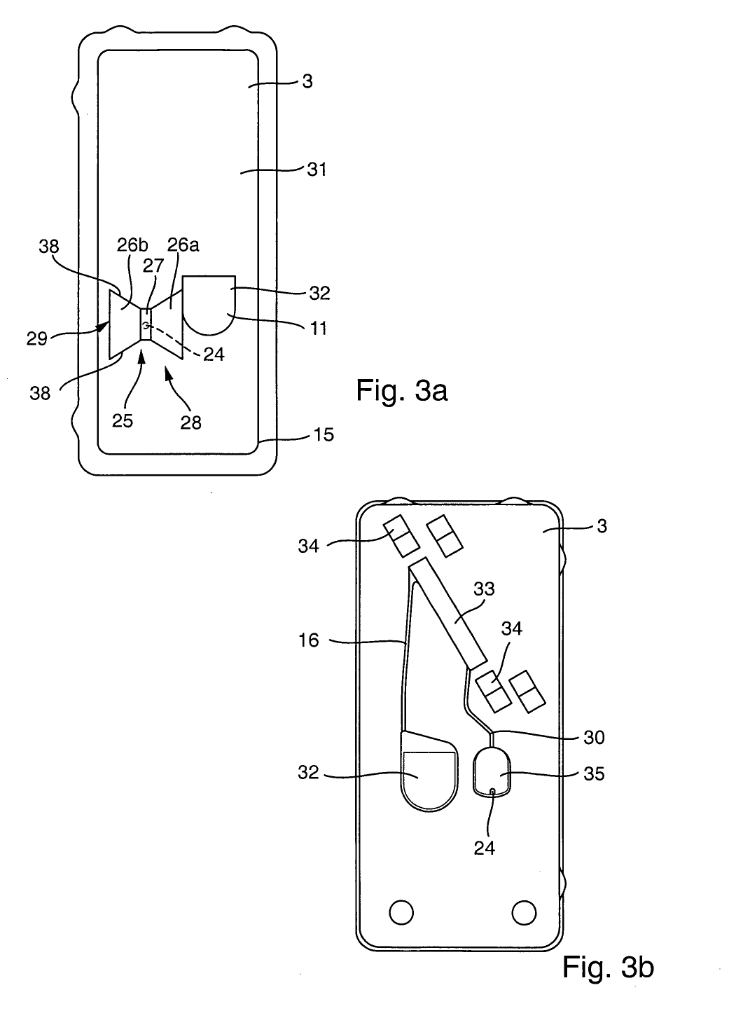

FIGS. 3A-3D show various schematic views of a further test element, according to embodiments of the present disclosure;

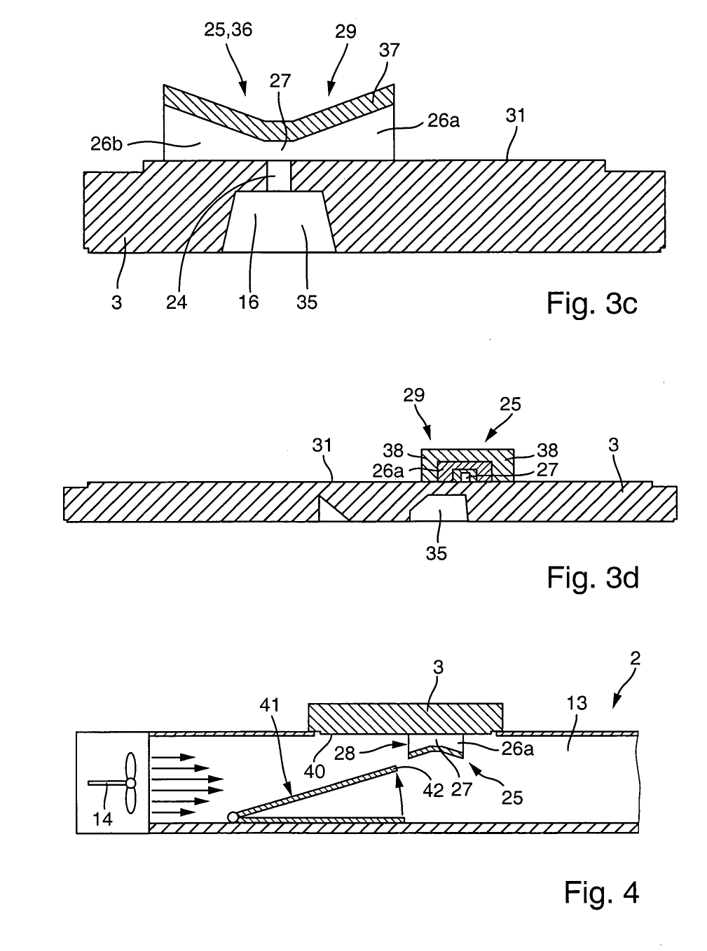

FIG. 4 shows a schematic view of the test element inside an analysis system, according to embodiments of the present disclosure.

### DETAILED DESCRIPTION

Embodiments of the present disclosure relate to a test element and an analysis system for the analysis of a body fluid sample for an analyte contained therein, and a method for controlling the movement of a liquid contained in a channel of a test element for the analysis of a body fluid. The analysis of the body fluid is typically performed for medical purposes. On this basis, embodiments of the present disclosure are configured for providing a test element and an analysis system having test elements which allow improved liquid transport.

This is achieved by a test element for the analysis of a body fluid sample for an analyte contained therein. Some embodiments include an analysis system for analyzing a body fluid sample, while some include a method for controlling the movement of a liquid contained in a channel of a test element for the analysis of a body fluid sample.

The test element according to some embodiments of the present disclosure comprise an analysis function channel, which is enclosed by the housing, an airflow channel having two expansion sections, and a narrow section lying between them, and a connection channel, which connects the narrow section of the airflow channel and the analysis function channel so that an air exchange occurs between them. An airflow flowing through the airflow channel generates a partial vacuum (reduced pressure) via the air exchange connection of the connection channel, which has an effect on the analysis function channel. A body fluid sample located in the analysis function channel is sucked in by this generated suction effect. By controlling the partial vacuum, it is thus possible to influence the air conditions inside the analysis function channel, in particular the pressure prevailing in the channel.

Ambient air of the environment of the test element flows through the airflow channel when the rotatable test element moves. The airflow is generated by the relative movement of the test element to the environment. Controlling the rotation of the test element, in particular by control of the rotational velocity and the rotational direction, the pressure in the analysis function channel can be set in a defined manner. An additional air supply is thus not necessary. External air pumps or blowers can be dispensed with. The analysis systems in which the test element can be inserted and rotated can accordingly be constructed compactly and simply, because space is not required for a blower or an additional controller. The analysis systems can thus be produced cost-effectively.

In some embodiments, the test element rotates around a rotational axis, which typically extends through the test element. The airflow channel is oriented such that the ambient air can flow through the airflow channel during the rotation of the test element. For this purpose, the airflow channel has a tangential component to the rotational axis. By means of a corresponding configuration of the airflow channel, it can be established how great the maximum generated partial vacuum in the connection channel and in the analysis function channel is at an established rotational velocity. The greater the tangential component of the flow channel, the greater the partial vacuum which can be generated in the connection channel. The greatest possible partial vacuum can be generated with a tangential orientation of the airflow channel (surface normal of the opening surface is parallel to the velocity vector).

The flow channel is typically positioned on the upper side and/or the lower side of the housing of the test element so that the ambient air can flow as unrestrictedly and efficiently as possible through the flow channel. For example, the airflow channel can be positioned so that the opening surface of an air intake of the airflow channel is positioned perpendicular to the upper side of the housing of the test element. The air intake may be at a maximum in this configuration. The airflow channel can run (substantially) parallel to the upper side of the housing, for example.

The airflow channel may be positioned on the upper side or lower side of the test element housing in such a manner that its narrow section is positioned above or below an analysis function channel, respectively. The connection channel, which connects the analysis function channel and the narrow section of the airflow channel, is as short as possible in this case, so that the air exchange between the two channels is particularly good and a greater partial vacuum can already be generated at low flow velocities of the air flowing through the airflow channel. Of course, applications are also conceivable in which a limited partial vacuum is to be generated in the analysis function channel, or a detectable effect on the liquid positioned in the analysis function channel is to be achieved only at a predefined (relatively high) speed of the test element. In this case, the connection channel is elongated, so that the narrow section of the airflow channel is farther away from the analysis function channel. By means of suitable dimensioning and positioning of the airflow channel in relation to the analysis function channel, the partial vacuum to be achieved can thus be predetermined.

In some embodiments, the analysis function channel comprises at least one microfluidic channel section. A microfluidic channel section or channel in the meaning of embodiments of the present disclosure typically have a wall spacing of at most approximately 300 μm. in some embodiments, the cross-section is not round. In the case of an oblong, rectangular cross-sectional shape, the longer dimension (depth) of the channel can have much greater values. It is decisive that the spacing, more specifically, the shortest distance of adjacent walls has at most the specified value. Of course, in addition to the microfluidic channel, the test element can also contain channels or channel sections having greater dimensions, in which no or only very small capillary forces have an effect on the liquid. It is decisive that at least one channel, typically a plurality of channels or channel sections of the test element, maintains the specified dimensions.

Many test elements have feed openings, which are connected to a channel, such as an analysis function channel, for feeding a liquid into a channel of a test element in a predetermined direction and/or at a predefined velocity. During dosing of the liquid into the feed opening of the channel, the problem frequently arises that air bubbles are formed, by which the absorption of the liquid (liquid sample such as blood) is interfered with or obstructed. The further transport of the liquid can also be negatively influenced by poor ventilation of the channel, for example, by an uneven or time-delayed further flowing of the liquid in the channel.

In order to improve the ventilation in the channel, the ventilation channel may be positioned in proximity to the feed opening to implement an air-release (ventilation) of the channel. The ventilation channel is hydrophobic, so that no liquid is transported therein. It may be used in particular for the purpose of transporting air, which is displaced by feeding the liquid into the channel, or another gaseous medium out of the channel. Because the housing of the test element is typically made of plastic, the channel walls in the ventilation channel are hydrophobic in any case. A special hydrophobization of the plastics which are typical in analysis technology for medical purposes is therefore not necessary.

In the context of the present disclosure it has been established that not only the movement of the body fluid sample in the channel can be controlled by the suction action, but rather also the ventilation of the channel can be improved. According to some embodiments of the present disclosure, the connection channel between the analysis function channel and the airflow channel is a ventilation channel. The air-release action of the ventilation channel is enhanced by the suction action described in the connection channel.

According to embodiments the present disclosure, a process may be utilized for the ventilation of a sample analysis channel. The test element having a housing has an airflow channel having two expansion sections and a narrow section positioned between them and a ventilation channel, which is positioned between the narrow section and the analysis function channel and through which an air exchange occurs. According to embodiments of the present disclosure, an airflow is generated for the ventilation, the airflow is deflected through the airflow channel in such a manner that the airflow passes through the narrow section. Additionally, a partial vacuum is generated in the ventilation channel by the airflow, which sucks out a gaseous medium present in the analysis function channel. The introduction of a liquid into the analysis function channel is thus made easier. An unobstructed and bubble-free feed of a liquid into the analysis function channel is made possible.

For controlling the movement of a liquid contained in a channel of a test element for analysis of a body fluid sample, a process may be performed according to embodiments of the present disclosure, which may run automatically or in an automated manner. In some embodiments, the test element comprises a housing and an analysis function channel enclosed by the housing, an airflow channel having two expansion sections and a narrow section positioned between them, and a connection channel, which is positioned between the narrow section and the analysis function channel. The narrow section and the analysis function channel have an air exchange connection to one another by means of the connection channel. The expansion sections have a greater cross-sectional area in comparison to the narrow section, which increases in the direction away from the narrow section.

According to embodiments of the present disclosure, an airflow is generated to control the movement of the sample in that the test element is rotated. The airflow is guided through the airflow channel in such a manner that the airflow flows through the narrow section. In some embodiments, the airflow flows through one of the two expansion sections into the airflow channel, passes the narrow section, and flows out through the other expansion section.

In a further process, a partial vacuum is generated in the connection channel, which connects the analysis function channel and the narrow section of the airflow channel to one another. The partial vacuum has an effect on the analysis function channel in such a manner that the liquid sample contained therein is influenced in its location. In this manner, the movement of the liquid sample can be changed. Not only the position of the liquid sample (direction of its movement) is controllable, but rather also the flow velocity of the liquid sample in the analysis function channel. The liquid can be accelerated or decelerated. The liquid can be slowed in such a manner that it comes to a standstill.

The automatic method according to embodiments of the present disclosure may comprise a process, in which a liquid sample is dosed through a sample feed opening into the analysis function channel. In some embodiments, this is performed before the other portions of the method.

Referring now to the drawings, FIG. 1 shows an embodiment of the analysis system 1 according to embodiments of the present disclosure for analyzing a body fluid sample for an analyte contained therein having an evaluation device 2 and a test element 3, which may be controllable and/or disposable, and which rotates around a rotational axis. The test element 3 is held in a rotatable mounting (not shown), which is part of the evaluation device 2.

The evaluation device 2 has a drive 4 for the movement of the test element 3 around the rotational axis. The transport of a liquid, such as a sample liquid, in the test element 3 is externally controlled by the rotational movement. The drive 4 is controlled by a drive controller 5 in such a manner that the rotational direction and the rotational velocity are controlled. In this manner, the flow velocity, the flow direction, and the dwell time of liquids in specific sections of the test element can be established.

The evaluation device 2 includes a measuring station 6 for measuring a measuring variable, which is characteristic for the analysis result, at a measuring zone 19 of the test element 3. The measuring station typically comprises an optical measuring apparatus 7 and an evaluation unit 8 for determining the characteristic measuring variable. The optical measuring apparatus 7 typically includes a measuring device for fluorescence measurement with a location-sensing detection, in which the illumination on the measuring zone of the test element 3 and/or the excitation of optically detectable markings in the test zone by means of an LED or a laser is performed by a two-dimensional evaluation optic, for example. The detection is performed, for example, using a charge coupled device (CCD) and/or a complementary metal oxide semiconductor (CMOS) optic. Of course, other optical methods can also be used. In some embodiments, the test element 3 is positioned in a measuring position for the measurement. Thus, the test element 3 is at rest. However, it is also possible to perform the measurement during a movement of the test element 3. This is possible, for example, at low rotational speed (up to approximately 600 revolutions per minute (RPM)).

In a preferred embodiment, the evaluation device 2 can optionally be equipped with a dosing station 9, which has a dosing needle 10 to apply a liquid in the test element 3. The dosing station 9 can comprise one or more liquid reservoirs (not shown here) for this purpose, in which various liquids can be stored, for example. These liquids, which may be dosed using the dosing station 9, may be, for example, the sample liquid such as blood containing the analyte, a washing liquid, a washing buffer, or a flushing liquid, which can be dosed into a feed opening 11 and/or a feed opening 12.

The test element 3 is typically located in a dosing position in the evaluation device 2, while a liquid is dosed into one or more of the feed openings 11, 12 by the dosing station. A manual feed of a liquid into the feed openings 11, 12 by the user (for example, via pipette) is also conceivable.

In some embodiments, the evaluation device 2 comprises a receptacle space 13, in which the test element 3 rotates. In still some embodiments, the evaluation device 2 includes an external flow source, such as a fan 14, which can typically be part of a temperature control unit. The temperature control unit is used for heating the test element 3, the liquid sample, and/or another liquid (liquid medium), which can be applied in the test element 3. The fan 14 can generate an external airflow, whose use is explained in greater detail with reference to FIG. 4.

FIG. 2 shows an embodiment of a test element 3, which has a housing 15 having a substrate and a central hole, which is used as a drive hole for the mounting in the evaluation device 2. In addition to the substrate, the disc-shaped test element 3 also contains a cover layer (not shown here). The cover layer can also fundamentally carry fluidic structures; however, the cover layer may have openings for the feed of liquids or valve openings. The housing 15 of the test element has fluidic or microfluidic and chromatographic structures. The sample liquid, in particular whole blood, is fed to the test element 3 via the feed opening 11.

The test element 3 has two at least partially microfluidic analysis function channels 16, one being a sample analysis channel 17 and the other being a flushing liquid channel 18. The flushing liquid channel 18 extends from the feed opening 12 to a collection zone 23. In the flow direction, the sample analysis channel 17 includes the sample feed opening 11 at its beginning and a measuring zone 19 at its end. In between, the sample analysis channel 17 comprises a microfluidic primary channel section 20, a capillary stop 21, a microfluidic secondary channel section 22, and the collection zone 23, which is a collection chamber. The capillary stop 21 can be implemented as a geometric valve or hydrophobic barrier. The secondary channel section 22 guides a sample quantity measured by the capillary stop 21, which is controlled, for example, by centrifugal forces by means of the rotational velocity of the test element 3.

It can be seen from FIG. 2 that the test element 3 comprises a plurality of airflow channels 25, which are each connected via a connection channel 24 to the analysis function channel 16. The airflow channel 25 includes expansion sections 26a, 26b and a narrow section 27, which is located between the expansion sections 26a, 26b. The expansion sections 26a, 26b each have a cross-sectional area which increases in comparison to the narrow section in the direction away therefrom. The connection channel 24 is connected to the narrow section 27 in such a manner that an air exchange is provided between the analysis function channel 16 and the airflow channel 25.

The design construction of the airflow channel 25 fundamentally corresponds to a Venturi nozzle 28, the Venturi nozzle 28 together with the connection channel 24 form a Venturi configuration 29. The airflow channel 25 accordingly operates according to the Venturi principle, according to which the flow velocity in the narrow section is significantly greater than in the expansion sections 26a, 26b. In this manner, a partial vacuum is generated in the connection channel 24, the vacuum also acts in the analysis function channel 16 based on the air exchange connection and generates a suction effect for controlling a liquid inside the analysis function channel 16.

The sample analysis channel 17 has a plurality of Venturi configurations 29a to 29d for controlling the sample liquid inside the channel. The Venturi configurations 29a to 29c are used for controlling a defined further flow of the sample liquid. Not only can the flow velocity be increased, it can also be slowed so that the liquid comes to a standstill (stasis). This can be advisable in particular if reagents in dry form are to be dissolved in the primary channel section 20. The Venturi configurations 29a to 29c thus form so-called fluid stop-and-go switches. It is also possible to cause the liquid to flow opposite to the processing-related flow direction by means of the suction action induced by the airflow channel 25.

While the Venturi configurations 29a to 29c are used for influencing the velocity and/or direction of the liquid, the Venturi configuration 29d additionally has a so-called gatekeeper function. The flow of the liquids further out of the collection zone 23 through the channel section 19a to the measuring zone 19 is guided by the Venturi configuration 29d. This airflow channel 25 thus also has a kind of shunt function; it ensures that, for example, a flushing liquid cannot flow out of the flushing liquid channel 18 into the secondary channel section 22. The airflow channel 25 of the Venturi configuration 29d is positioned directly above the channel section 19a, so that the connection channel 24 is very short. The narrow section 27 can be nearly arbitrarily short. It must only allow a connection of the airflow channel to the connection channel 24 for the air exchange.

Furthermore, the Venturi configuration 29d allows a flow of the liquid opposite to the centrifugal force, namely in the direction of the rotational axis. The suction force generated by the (partial) vacuum in the channel section 19a must be greater than the centrifugal force generated due to the rotation. However, the suction action can be supported by capillary forces. The force generated by the partial vacuum (reduced pressure) thus represents a third force component (in addition to the centrifugal forces and capillary forces) for controlling the liquid movement.

FIG. 2 also shows that the airflow channel 25 of the Venturi configuration 29, 29a to 29d are positioned in such a manner that they have a tangential component to the rotational direction. In this manner, ambient air can flow into the airflow channel 25 during the rotation of the test element 3. In this embodiment, the airflow channels are therefore positioned on the upper side of the housing 15 of the test element 3, from which the liquids are also dosed into the feed openings 11, 12.

A ventilation channel 30 is positioned at the beginning of the analysis function channel 16 behind the feed opening 11. This hydrophobic channel is used for ventilating the analysis function channel 16, so that gaseous medium displaced by the inflowing liquid can escape. The Venturi configuration 31, which comprises the ventilation channel 30, includes an airflow channel 25 (as described above). In this manner, the ventilation of the analysis function channel 16 is significantly improved, because the air located in the channel is suctioned out and no air bubbles can be formed.

FIGS. 3A to 3D show a further embodiment of the test element 3 according to embodiments of the present disclosure, which is not implemented here as a round disc, but rather as a rectangular plate. This plate-shaped disc is inserted into a mounting of the evaluation device 2, so that it can rotate around a rotational axis.

FIG. 3A shows the upper side 31 of the test element 3 having a feed opening 11 and a Venturi nozzle 28, which includes an airflow channel 25. The two expansion sections 26a, 26b and the narrow section 27 positioned between them are clearly shown, wherein the flow speed of the air flowing through the narrow section 27 is increased.

FIG. 3B shows a section view of the test element 3 from below. The feed opening 11 leads to a feed zone 32, to which an analysis function channel 16 is connected. The analysis function channel 16 leads into a cuvette 33, in which the sample liquid is stored and analyzed by means of an optical evaluation. Therefore, the test element 3 comprises two mirrors 34, so that light can be conducted through the cuvette 33. A transmission measurement of the sample liquid contained in the cuvette 33 is possible by this design.

On the end which is opposite to the inlet of the sample liquid, the cuvette 33 has a ventilation channel 30, which generates an air exchange connection between the cuvette 33 and an air chamber 35. The air chamber 35 is designated as a “compressed-air accumulator” (windkessel). The air chamber 35 is connected via a connection channel 24 to the narrow section 27 of the airflow channel 25. A simple, reliable, and controlled ventilation of the cuvette 33 can be performed in this manner.

FIG. 3C shows the Venturi configuration 29 as a sectional illustration through the test element 3 in detail. The air chamber 35 implemented as a windkessel is clearly shown, the air chamber 35 is connected via the connection channel 24 to the Venturi configuration 29. The airflow channel 25 is positioned as a funnel 36, whose base surface is formed by the upper side 31 of the housing 15 of the test element 3. The upper side of the funnel 36 is covered by a cover plate 37, which inclines toward the narrow section 27 of the airflow channel 25. The two side walls 38 (shown in FIG. 3A) of the funnel-shaped expansion section 26a, 26b, which are shown in FIG. 3A, are positioned in relation to one another in such a manner that they spread apart away from the narrow section 27.

It is clear to those skilled in the art that the roof or cover plate 37 of the funnel 36 does not necessarily have to be inclined. Of course, it can also be positioned parallel to the upper side 31 of the housing 15.

The expansion sections 26a, 26b expand from the narrow section 27 in the longitudinal direction, typically continuously. Of course, it is also possible that the expansion sections expand in the form of steps or a staircase, a plurality of steps typically being provided. It is also conceivable, however, that the expansion in the longitudinal direction is performed in only one step, which is formed between the expansion section 26a, 26b and the narrow section 27.

FIG. 3D shows a section view through the airflow channel 25 transversely to the longitudinal direction. The various sections of the airflow channel 25 with the narrow section 27 in the middle and the expansion section 26a lying in front of it are clearly shown.

FIG. 4 shows a detail view of the evaluation device 2 from FIG. 1. Here, the receptacle space 13 of the evaluation device 2 having a test element 3 mounted therein is shown. The test element 3 has an airflow channel 25, which functions as a Venturi nozzle 28, on the lower side 40 of the housing 15.

The evaluation device 2 typically includes an air guiding element, which guides the airflow generated by the fan 14 in the direction of the airflow channel 25 of the test element 3 in such a manner that the airflow flows through the airflow channel, typically when the test element 3 is located in a through-flow position. Typically the air guiding element is positioned movably. The airflow generated by the fan can flow in the airflow channel 25 additionally or alternatively to the airflow of the ambient air, which is generated in the channel by the rotation of the test element.

An embodiment of such an air guiding element is shown in FIG. 4. The air guiding element 41 is an adjustable control flap 42, which can be pivoted around a pivot axis, for example. The air flowing into the Venturi configuration 29 can thus be controlled and compressed. The airflow generated by the fan 14 is then conducted very specifically into the intake of the airflow channel 25. Of course, other air guiding elements 41 are also conceivable, such as flaps that are not pivotable vertically like the control flap 42, but rather are pivoted or rotated horizontally, such as around a vertical axis. It is also possible to incline, raise, and/or lower the base or the cover of the receptacle space 13, in order to change the space available for the externally generated airflow. The suction action that is generated by the Venturi nozzle 28 in the analysis function channel 16 of the test element 3 may also be changed and controlled in this manner.

Using an external fan 14, which can be part of a temperature control unit for heating the test element or the liquid sample, for example, it is possible to generate an airflow, which flows through the airflow channel 25 and thus controls a liquid, even at a standstill of the test element 3. A liquid is then moved in the analysis function channel 16 of the test element 3 only by the suction action generated as a result of the generated partial vacuum in the Venturi configuration 29 or by capillary forces. A centrifugal force based on the rotation of the test element does not exist in this case.

The through-flow position, in which the test element 3 is located when an externally generated airflow flows through the airflow channel 25, is typically similar to the dosing position, in which a liquid is dosed into at least one of the feed openings 11, 12. In this manner, dosing can be supported and simple and reliable ventilation of the analysis function channel 16 can be achieved. However, it is also possible that the test element 3 moves while the airflow of the fan acts on the Venturi configuration 29. Depending on the control and guiding of the external airflow, the partial vacuum caused by the rotational movement in the analysis function channel 16 can be increased or decreased to change or control the movement of the liquid in the analysis function channel 16.

Therefore, at least the following is claimed:

1. A test element for analysis of a body fluid sample for an analyte contained therein comprising:
a housing and an analysis function channel, which is enclosed by the housing,
an airflow channel having two expansion sections and a narrow section positioned between these expansion sections,
wherein:
the test element is implemented in such a manner that it is rotatable in an evaluation device around a rotational axis, in order to control transport of the body fluid sample in the test element,
the test element is rotatable around the rotational axis,
the airflow channel is oriented in such a manner that ambient air can flow through the airflow channel during rotation of the test element,
the expansion sections each have a cross-sectional area increasing in comparison to the narrow section in a direction away therefrom, and
a connection channel is positioned between the narrow section and the analysis function channel in such a manner that the narrow section and the analysis function channel have an air exchange connection to one another, so that a partial vacuum has an effect on the analysis function channel, the partial vacuum is generated via the ambient air flowing through the airflow channel during the rotation of the test element.

2. The test element according to claim 1, wherein the analysis function channel comprises at least one microfluidic channel section.

3. The test element according to claim 1, wherein the rotational axis around which the test element is rotatable extends through the test element.

4. The test element according to claim 1, wherein the airflow channel is positioned according to at least one of the following: on an upper side of the test element and on a tower side of the housing of the test element.

5. The test element according to claim 1, wherein the partial vacuum acts on a liquid in the analysis function channel in such a manner that a movement of the liquid in the analysis function channel is thus controllable.

6. The test element according to claim 5, wherein the movement of the liquid in the analysis function channel occurs according to at least one of the following: in a predetermined direction and at a predefined velocity.

7. The test element according to claim 1, wherein the expansion section adjoining the narrow section expands continuously.

8. The test element according to claim 1, wherein the connection channel is a ventilation channel.

9. The test element according to claim 8, wherein the ventilation channel is a hydrophobic ventilation channel.

10. The test element according to claim 1, wherein the analysis function channel is a sample analysis channel, which includes a sample feed opening and a measuring zone.

11. An analysis system for analyzing a body fluid sample for an analyte contained therein, comprising
a test element, which has a housing and an analysis function channel, which is enclosed by the housing, and
an evaluation device having a measuring station for measuring a measuring variable, which is characteristic for an analysis result, on a measuring zone of the test element and having a rotatable mounting for holding the test element,
wherein:
the test element mounted in the rotatable mounting is rotatable around a rotational axis in order to control transport of the body fluid sample in the test element,
the test element has an airflow channel having two expansion sections and a narrow section positioned between these expansion sections, wherein the expansion sections haying a cross-sectional area which increases in comparison to the narrow section in a direction away therefrom,
a connection channel is positioned between the narrow section and the analysis function channel in such a manner that an air exchange connection is formed,
the airflow channel is oriented in such a manner that ambient air can flow through the airflow channel during rotation of the test element, and
a partial vacuum generated by means of the ambient air flowing through the airflow channel has an effect on the analysis function channel.

12. The analysis system according to claim 11, wherein the evaluation device comprises a fan, which generates an airflow, and the airflow can additionally flow through the airflow channel to generate a partial vacuum, which has an effect on the analysis function channel.

13. The analysis system according to claim 12, wherein the fan is part of a temperature control unit, which is used to heat at least one of the following: the test element and a liquid medium.

14. The analysis system according to claim 12, further comprising an air guiding element, which guides the airflow generated by the fan in the direction of the airflow channel of the test element in such a manner that the airflow flows through the airflow channel when the test element is in a through-flow position.

15. The analysis system according to claim 14, wherein the air guiding element is a movable air guiding element.

16. The analysis system according to claim 11, wherein, in a dosing position of the test element in the analysis system, a liquid is dosed into a sample feed opening of the analysis function channel by means of a dosing station, and the dosing position is typically identical to a through-flow position of the test element, in which the test element is positioned in the evaluation device in such a manner that an airflow can flow through the airflow channel.

17. A method for controlling movement of a liquid contained in a channel of a test element for analysis of a body fluid sample, the test element is implemented in such a manner that it is rotatable in an evaluation device around a rotational axis in order to control transport of the body fluid sample in the test element, and the test element comprises:
a housing,
an analysis function channel enclosed by the housing,
an airflow channel having two expansion sections and a narrow section positioned between these expansion sections, the expansion sections each having a cross-sectional area which increases in comparison to the narrow section in a direction away therefrom, and
a connection channel, which is positioned between the narrow section and the analysis function channel in such a manner that the narrow section and the analysis function channel are connected to one another by an air exchange connection,
the method comprising:
generating an airflow by rotation of the test element,
guiding the airflow through the airflow channel in such a manner that the airflow flows through the narrow section, and
generating a partial vacuum in the connection channel, which acts on the analysis function channel in such a manner that the body fluid sample contained therein is influenced in its location.
